(19) United States
(12) Patent Application Publication
(10) Pub. No.: US 2004/0043952 A1
(43) Pub. Date: Mar. 4, 2004

(54) Multifunctional polyamines for delivery of biologically-active polynucleotides

(75) Inventors: Edmund J. Niedzinski, Vacaville, CA (US); Michael Bennett, EI Sobrante, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Genteric, Inc., Alameda, CA

(21) Appl. No.: 10/449,852

(22) Filed: May 30, 2003

Related U.S. Application Data
(60) Provisional application No. 60/384,514, filed on May 31, 2002.
(60) Provisional application No. 60/385,234, filed on May 31, 2002.

(51) Int. Cl. A61K 48/00; A61K 31/44; A61K 31/40; A61K 31/24; C07C 279/10; C07C 229/40

(52) U.S. Cl. 514/44; 514/357; 514/408; 514/534; 560/39; 560/34

(57) ABSTRACT

The present invention relates, inter alia, to pentaerythritol lipid derivatives that are useful for the intracellular delivery of nucleic acids. Such pentaerythritol lipid derivatives are useful for the preparation of transfection complexes (such as liposomes, lipoplexes, and other lipid vesicles) that can be used to deliver nucleic acids into mammalian cells.

### CROSS-REFERENCE TO RELATED APPLICATIONS

The present application claims priority to U.S. Provisional Patent Application Nos. 60/384,514 and 60/385,234, both filed on May 31, 2002, the disclosures of which are hereby incorporated by reference in their entireties for all purposes.

### BACKGROUND OF THE INVENTION

The present invention relates, inter alia, to pentaerythritol lipid derivatives that are useful for the intracellular delivery of nucleic acids. Such pentaerythritol lipid derivatives are useful for the preparation of transfection complexes (such as liposomes and other lipid vesicles) that can be used to deliver nucleic acids into mammalian cells.

The introduction of foreign nucleic acids and other molecules is a valuable method for manipulating cells and has great potential both in molecular biology and in clinical medicine. Many methods have been used for insertion of endogenous nucleic acids into eukaryotic cells. Genetic material can be introduced into cells to express an encoded protein which is deficient or defective. The use of such technology allows for the treatment of genetic based diseases. Gene transfer entails distributing nucleic acids to target cells and then transferring the nucleic acid across a target cell membrane in a form that can function in a therapeutic manner. Of the many methods used to facilitate entry of DNA into eukaryotic cells, cationic liposomes are among the most efficacious and have found extensive use as DNA carriers in transfection experiments. Cationic lipids themselves are known to bind to polynucleotides and to facilitate their intracellular delivery into mammalian cells. Nucleic acid is negatively charged and when combined with a positively charged lipid forms a complex that is suitable for formulation and cellular delivery. The use of cationic lipid carriers for transfection is well established. However, their ability to mediate transfection is not well understood.

The precise way in which nucleic acids and cationic lipids interact and the structure formed before and during the transfection process are not well known. It is commonly believed that the nucleic acids are entrapped within a lipid bilayer, which is the classic definition of a “liposome.” There is also a belief, however, that the nucleic acid does not become entrapped, but forms some other sort of aggregate with the cationic lipids. It has also been reported that liposome-DNA aggregate size and shape are a function of the ratio of the amount of DNA to that of cationic lipid. It has been concluded that DNA binds to the outer surface of liposomes, which then cluster into irregular spherical aggregates. Plasmid length had no effect on binding of data to liposomes and the structure of the liposome-DNA complex is believed to change at charge neutrality, while the DNA becomes organized into a very compact structure that is evidently quite different from a liposome. It has been concluded that the liposome probably uses at least two pathways to introduce DNA into cells: fusion with the plasma membrane and endocytosis.

The delivery and expression of a transfected gene constitute a complex process that includes steps involving transfection complex (i.e., lipoplex) formulation, cellular internalization, endosomal escape, and nuclear localization. Incorporation of cationic lipids in the cytoplasmic membrane can occur by cytoplasmic fusion or translocation after lipoplex uptake. Cellular processes can be inhibited by the incorporation of positively charged lipids into the plasma membrane. This incorporation can lead to cell dysfunction and possibly cell death. Thus, although there are benefits to cationic lipid facilitated gene transfer, there are also deleterious effects of lipidic salts on cellular processes. In fact, the long-term administration of cationic lipoplexes has been shown to elicit inflammatory responses and cytotoxicity.

Lipid-associated cytotoxicity has been attributed to the inhibition of protein kinase C activity by cationic lipids after internalization of the lipoplex. This is presumably a consequence of cationic lipid incorporation into the plasma membrane. In addition, transfection is attributed to the formation of transmembrane pores. There are also resultant disruptions of signal transduction and gene regulation processes which impair cellular function. It is thought that enhanced clearance of the cationic lipids might alleviate lipid-associated cytotoxicity.

As such, there exists a need for lipids that are effective in facilitating intracellular delivery of genetic material, but that have reduced lipid-associated cytotoxicity. The present invention fulfills this and other needs.

### SUMMARY OF THE INVENTION

The present invention provides multifunctional polyamine (MFP) compounds that facilitate the delivery of biologically active polynucleotides for numerous therapeutic purposes. The MFP compounds of the present invention contain two distinct chemical domains, a nucleic acid binding domain (e.g., a DNA binding domain) and a hydrophobic domain, that are linked through ester bonds to a symmetrical, poly-hydroxylated core structure. Specifically, the DNA binding domain is constructed by adding a compound or molecule with an amine functional group to an acrylate, resulting in a β-amino ester. The resulting MFP compounds can be formulated with polynucleotides and delivered, in vitro or in vivo, to enhance the desired therapeutic effect of the polynucleotide. The strategy that is used to synthesize these chemicals can be easily adapted to allow for the introduction of structurally diverse DNA binding domains. The development of this structural design has yielded a panel of MFP compounds that are more active and less toxic than the compounds that are currently used to facilitate the polynucleotide delivery.

More particularly, the present invention provides multifunctional polyamine (MFP) compounds that are useful as transfection agents. It has been found that the MFP compounds function as versatile transfection lipids. The synthetic versatility of the core structure allows for the introduction of structurally diverse hydrophobic and nucleic acid-binding domains via ester bond formation. Advantageously, the low cytotoxicity of the MFP compounds, due in part to their susceptibility to ester hydrolysis under physiological conditions, greatly facilitates their metabolism. The rapid clearance of such MFP compounds of the present invention is an important factor in maintaining normal cellular function as the inadvertent binding of cationic lipids to protein kinase C (and other essential cellular enzymes) is regarded as an origin of cytotoxicity (see, Farhood et al., Biochim. Biophys. Acta., 1111:239-246 (1992)).

As such, in one embodiment, the present invention provides a MFP compound, the MFP compound having the following structure:

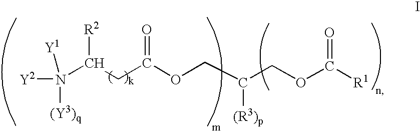

or a pharmaceutically acceptable salt thereof.

In Formula I, each R¹ is independently selected from the group consisting of optionally substituted C₈-C₂₄ alkyl and optionally substituted C₈-C₂₄ alkenyl; each R² and R³ is independently selected from the group consisting of hydrogen, optionally substituted C₁-C₈ alkyl, optionally substituted arylalkyl, optionally substituted C₁-C₈ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl; each Y¹ and Y² is independently selected from the group consisting of hydrogen, optionally substituted C₁-C₆ alkyl, optionally substituted arylalkyl, optionally substituted C₁-C₆ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl; each Y³, if present, is independently selected from the group consisting of hydrogen, optionally substituted C₁-C₈ alkyl, optionally substituted arylalkyl, optionally substituted C₁-C₈ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl; m is an integer selected from the group consisting of 1, 2, 3 and 4, n is an integer selected from the group consisting of 0, 1, 2 and 3, and p is an integer selected from the group consisting of 0 and 1, wherein the sum of m, n and p is 4; each k is an integer independently selected from the group consisting of 1, 2, 3, 4 and 5; and each q is an integer independently selected from the group consisting of 0 and 1; or, each R², Y¹ and the atoms to which they are bound, join to form an optionally substituted 5- or 6-membered heterocyclic ring. It is noted that the foregoing substituents are selected such that if m is 2, n is 2, q is 0 and Y¹ is hydrogen, then Y² is not an optionally substituted aminoalkyl.

In a preferred embodiment, each q is 1 and the compound is a pharmaceutically acceptable salt. In a preferred embodiment, the pharmaceutically acceptable salt is at least one quaternary nitrogen salt selected from the group consisting of a quaternary ammonium chloride, a quaternary ammonium iodide, a quaternary ammonium fluoride, a quaternary ammonium bromide, a quaternary ammonium oxyanion and a combination thereof. In a preferred embodiment, the pharmaceutically acceptable salt is a quaternary ammonium iodide.

In another embodiment, the present invention provides a transfection complex, the transfection complex comprising a nucleic acid and a compound of Formula I:

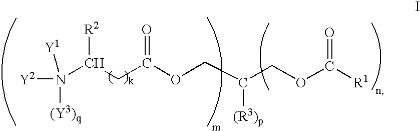

or a pharmaceutically acceptable salt thereof, wherein R¹, R², R³, Y¹, Y², Y³, m, n, k and q are as defined above and hereinbelow.

In a preferred embodiment, the nucleic acid is RNA or DNA. Examples of suitable nucleic acids include, but are not limited to, plasmid DNA, antisense RNA or DNA, etc. In a preferred embodiment, the transfection complex further comprises a second lipid or a mixture of lipids. Suitable lipids include, but are not limited to, DOSPA, DOPE, DMDHP, cholesterol and combinations thereof.

In another embodiment, the present invention provides a method for transfecting a cell with a nucleic acid, the method comprising contacting the cell with a transfection complex comprising the nucleic acid and a compound of Formula I:

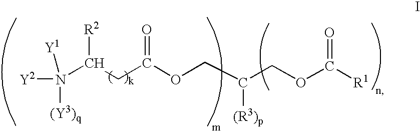

or a pharmaceutically acceptable salt thereof, wherein R¹, R², R³, Y¹, Y², Y³, m, n, k and q are as defined above and herein.

In another embodiment, the present invention provides a pharmaceutical composition, the pharmaceutical composition comprising (a) a transfection complex comprising a nucleic acid and a compound of Formula I:

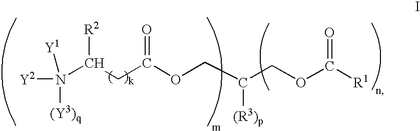

or a pharmaceutically acceptable salt thereof, wherein R¹, R², R³, Y¹, Y², Y³, m, n, k and q are as defined above and hereinbelow; and (b) a pharmaceutically acceptable carrier.

Other features, objects and advantages of the invention and its preferred embodiments will become apparent from the detailed description, figures and claims that follow.

### BRIEF DESCRIPTION OF THE DRAWINGS

FIG. 1 illustrates multifunctional polyamines in accordance with the present invention.

FIG. 2 illustrates a reaction scheme that can be used to synthesize the multifunctional polyamides of the present invention.

FIG. 3 illustrates the transfection of NIH-3T3 cells using two different multifunctional polyamines of the present invention.

### DETAILED DESCRIPTION OF THE INVENTION

It has been demonstrated that polynucleotides have the ability to treat a wide range of diseases. In fact, it has been found that the effective delivery of polynucleotides can lead to the in situ production of biologically active proteins, suppress the translation of RNA and elicit the production of therapeutic antibodies. In many applications, the poor bioavailability of polynucleotides requires that a chemical adjuvant be used to facilitate delivery. Chemical adjuvants, including the multifunctional polyamines of the present invention, are designed to protect and deliver the polynucleotide through various chemical interactions with the polynucleotide. Specifically, the multifunctional polyamines of the present invention exploit the ionic interaction of the positively charged nucleic acid-binding domain (e.g., DNA-binding domain) of the multifunctional polyamine with the negatively charged phosphates of the polynucleotides. Such interactions promote higher bioavailability, thereby allowing the polynucleotides to perform the previously discussed therapeutic effects.

The present invention demonstrates that the multifunctional polyamine (MFP) compounds function as versatile transfection lipids. The synthetic versatility of this core structure allows for the introduction of structurally diverse hydrophobic and nucleic acid-binding domains via ester bond formation. Advantageously, the low cytotoxicity of the MFP compounds, due in part to their susceptibility to ester hydrolysis under physiological conditions, greatly facilitates their metabolism. The rapid clearance of such cationic lipids is an important factor in maintaining normal cellular function as the inadvertent binding of cationic lipids to protein kinase C (and other essential cellular enzymes) is regarded as an origin of cytotoxicity (see, Farhood et al., Biochim. Biophys. Acta., 1111:239-246 (1992)).

A. Definitions

As used herein, the term “alkyl” denotes branched or unbranched hydrocarbon chains, preferably having from about 1 to about 8 carbons, such as methyl, ethyl, n-propyl, iso-propyl, n-butyl, sec-butyl, iso-butyl, tert-butyl, octa-decyl and 2-methylpentyl. Such alkyl groups can be optionally substituted with one or more functional groups including, but not limited to, hydroxyl, bromo, fluoro, chloro, iodo, mercapto or thio, cyano, alkylthio, heterocyclyl, aryl, heteroaryl, carboxyl, carbalkoyl, alkyl, alkenyl, nitro, amino, alkoxyl, amido, and the like to form alkyl groups such as trifluoro methyl, 3-hydroxyhexyl, 2-carboxypropyl, 2-fluoroethyl, carboxymethyl, cyanobutyl and the like.

The term “alkylene” refers to a divalent alkyl group as defined above, such as methylene (—CH₂—), propylene (—CH₂CH₂CH₂—), chloroethylene (—CHClCH₂—), 2-thiobutene (—CH₂CH(SH)CH₂CH₂—), 1-bromo-3-hydroxyl-4-methylpentene (—CHBrCH₂CH(OH)CH(CH₃)CH₂—), and the like.

The term “alkenyl” denotes branched or unbranched hydrocarbon chains containing one or more carbon-carbon double bonds, preferably having from about 8 to about 24 carbons. The alkenyl groups may have more than one site of unsaturation and the double bonds can be cis or trans.

The term “alkynyl” refers to branched or unbranched hydrocarbon chains containing one or more carbon-carbon triple bonds.

The term “aryl” denotes a chain of carbon atoms which form at least one aromatic ring having preferably between about 6 to about 14 carbon atoms, such as phenyl, naphthyl, and the like, and which may be substituted with one or more functional groups including, but not limited to, hydroxyl, bromo, fluoro, chloro, iodo, mercapto or thio, cyano, cyanoamido, alkylthio, heterocycle, aryl, heteroaryl, carboxyl, carbalkoyl, alkyl, alkenyl, nitro, amino, alkoxyl, amido, and the like to form aryl groups such as biphenyl, iodobiphenyl, methoxybiphenyl, anthryl, bromophenyl, iodophenyl, chlorophenyl, hydroxyphenyl, methoxyphenyl, formylphenyl, acetylphenyl, trifluoromethylthiophenyl, trifluoromethoxyphenyl, alkylthiophenyl, trialkylammoniumphenyl, amidophenyl, thiazolylphenyl, oxazolylphenyl, imidazolylphenyl, imidazolylmethylphenyl, and the like.

The term “acyl” denotes the —C(O)R group, wherein R is alkyl or aryl as defined above, such as formyl, acetyl, propionyl, or butyryl.

The term “alkoxy” denotes —OR, wherein R is alkyl.

The term “amido” denotes an amide linkage: —C(O)NR—, wherein R is hydrogen or alkyl.

The term “amino” denotes an amine linkage: —NR—, wherein R is hydrogen or alkyl.

The term “carboxyl” denotes —C(O)O—, and the term “carbonyl” denotes —C(O)—.

The term “carbonate” indicates —OC(O)O—.

The term “carbamate” denotes —NHC(O)O—, and the term “urea” denotes —NHC(O)NH—.

The term “nucleic acid” refers to a polymer containing at least two nucleotides. “Nucleotides” contain a sugar that is either deoxyribose or ribose, a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleotides are the monomeric units of nucleic acid polymers. A “polynucleotide” is distinguished here from an “oligonucleotide” by containing more than 80 monomeric units; oligonucleotides contain from 2 to 80 nucleotides. The term nucleic acid includes deoxyribonucleic acid (DNA) and ribonucleic acid (RNA). The term encompasses sequences that include any of the known base analogs of DNA and RNA.

DNA may be in the form of antisense, plasmid DNA, portions of a plasmid DNA, product of a polymerase chain reaction (PCR), vectors (P1, PAC, BAC, YAC, artificial chromosomes), expression cassettes, chimeric sequences, chromosomal DNA, or derivatives of these groups. RNA may be in the form of oligonucleotide RNA, tRNA (transfer RNA), snRNA (small nuclear RNA), rRNA (ribosomal RNA), mRNA (messenger RNA), antisense RNA, siRNA (small interfering RNA), ribozymes, chimeric sequences, or derivatives of these groups.

“Antisense” is a polynucleotide that interferes with the function of DNA and/or RNA. This may result in suppression of expression. Natural nucleic acids have a phosphate backbone, artificial nucleic acids may contain other types of backbones and bases. These include PNAs (peptide nucleic acids), phosphothionates, and other variants of the phosphate backbone of native nucleic acids. In addition, DNA and RNA may be single, double, triple, or quadruple stranded.

The term “recombinant DNA molecule” as used herein refers to a DNA molecule that is comprised of segments of DNA joined together by means of molecular biological techniques. “Expression cassette” refers to a natural or recombinantly produced polynucleotide molecule that is capable of expressing protein(s). A DNA expression cassette typically includes a promoter (allowing transcription initiation), and a sequence encoding one or more proteins. Optionally, the expression cassette may include trancriptional enhancers, noncoding sequences, splicing signals, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and noncoding sequences.

The term “gene” refers to a nucleic acid (e.g., DNA) sequence that comprises coding sequences necessary for the production of a polypeptide or precursor (e.g., myosin heavy chain). The polypeptide can be encoded by a full length coding sequence or by any portion of the coding sequence so long as the desired activity or functional properties (e.g., enzymatic activity, ligand binding, signal transduction, and the like) of the full-length or fragment are retained. The term also encompasses the coding region of a structural gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ nontranslated sequences. The sequences that are located 3′ or downstream of the coding region and which are present on the mRNA are referred to as 3′ nontranslated sequences. The term “gene” encompasses both cDNA and genomic forms of a gene. A genomic form or clone of a gene contains the coding region interrupted with noncoding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Upregulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

B. The Multifunctional Polyamine Compounds

In one embodiment, the present invention provides multifunctional polyamine compounds that facilitate nucleic acid delivery and yield biologically benign metabolites following transfection. The multifunctional polyamines, which are based on a pentaerythritol scaffold, have a hydrophobic domain and a nucleic acid-binding domain. In one embodiment, the present invention provides multifunctional polyamine compounds having the following general structure:

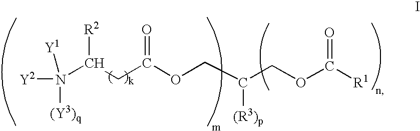

or a pharmaceutically acceptable salt thereof.

In Formula I, each R¹ is independently selected and is a functional group, including, but not limited to, optionally substituted C₈-C₂₄ alkyl and optionally substituted C₈-C₂₄ alkenyl. In a presently preferred embodiment, each R¹ is a functional group including, but not limited to, myristyl, oleyl, lauryl, steryl, palmityl and cholesteryl. In Formula I, each R² and R³ is independently selected and is a functional group, including, but not limited to, hydrogen, optionally substituted C₁-C₈ alkyl, optionally substituted arylalkyl, optionally substituted C₁-C₈ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl. In a presently preferred embodiment, each R² and R³ is independently selected and is a functional group, including, but not limited to, hydrogen, methyl, ethyl, isopropyl, isobutyl, secbutyl, hydroxymethyl, thiomethyl, carboxymethyl, guanidinopropyl, carbamoylmethyl, carbamoylethyl, benzyl, p-hydroxyphenylmethyl, 1-hydroxyethyl, 2-(methylthio)ethyl, 3-indolemethyl, carboxyethyl and aminobutyl. In another preferred embodiment, each R² and R³ is independently selected and is a functional group, including, but not limited to, hydrogen, methyl and isopropyl.

Each Y¹ and Y², in Formula I, is independently selected and is a functional group including, but not limited to, hydrogen, optionally substituted C₁-C₂₄ alkyl, optionally substituted C₈-C₂₄ alkenyl, optionally substituted arylalkyl, optionally substituted C₁-C₆ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl. In a presently preferred embodiment, each Y¹ and Y³ is independently selected and is a functional group including, but not limited to, methyl, ethyl, propyl and butyl; and each Y² is a independently selected and is a functional group including, but not limited to, N,N-dimethylethylamine, N,N-dimethylpropylamine and N,N-dimethylbutylamine.

In Formula I, each Y³, if present, is independently selected and is a functional group including, but not limited to, hydrogen, optionally substituted C₁-C₈ alkyl, optionally substituted arylalkyl, optionally substituted C₁-C₈ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl.

In Formula I, m is an integer having a value of 1, 2, 3 and 4, n is an integer having a value of 0, 1, 2 and 3, and p is an integer having a value of 0 or 1. It is noted that the values of m, n and p are selected such that the sum of m, n and p is 4.

Each k, in Formula I, is independently selected and is an integer including, but not limited to, 1, 2, 3, 4 and 5; and each q is independently selected and is an integer having a value of 0 or 1.

In certain embodiments, at least one of R², Y¹ and the atoms to which they are bound, join to form an optionally substituted 5- or 6-membered heterocyclic ring. In one preferred embodiment, at least of one R², Y¹ and the atoms to which they are bound, join to form a member selected from the group consisting of a pyrrolidine ring, a 4-hydroxypyrrolidine ring, a piperidine ring, a morpholine ring, a pyridine ring, a pyrazole ring, a pyrrole ring and an imidazolylmethyl ring.

It is noted that the values for m, n and q as well as the substituents for Y¹ and Y² are selected such that if m is 2, n is 2, q is 0 and Y¹ is hydrogen, then Y² is not an optionally substituted aminoalkyl.

In Formula I, the term “pharmaceutically acceptable salt” is meant to include salts of the active MFP compounds which are prepared with relatively nontoxic acids or bases, depending on the particular substituents found on the MFP compounds described herein. When the MFP compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt.

When the MFP compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to, those derived from inorganic acids like hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Certain specific compounds of the present invention contain both basic and acidic functionalities that allow the compounds to be converted into either base or acid addition salts.

In a preferred embodiment, the salts of the MFP compounds of the present invention exist as quaternized nitrogen salts such as quaternary ammonium salts including, but not limited to, quaternary ammonium chloride, quaternary ammonium iodide, quaternary ammonium fluoride, quaternary ammonium bromide, quaternary ammonium oxyanion and combinations thereof. As used herein, the term “quaternary ammonium salt” includes 1, 2, or 3 alkyl groups on the nitrogen atom. When more than one nitrogen exists in the multifunctional polyamine compounds, any or all nitrogens can be quaternized. For instance, 1, 2, 3 or more nitrogens can be quaternized.

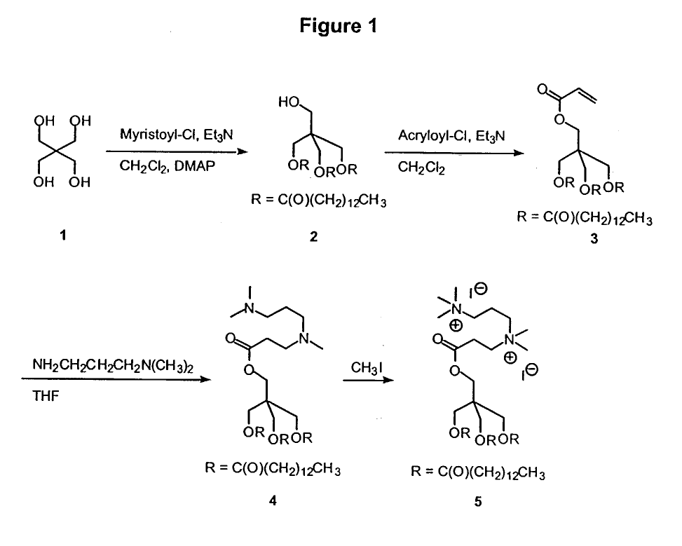

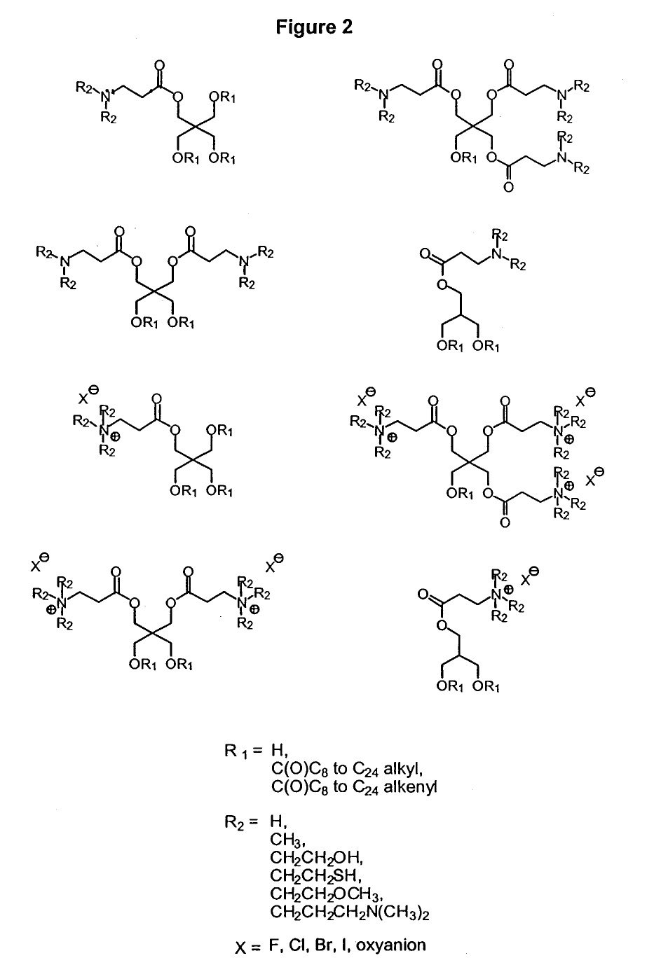

FIGS. 1 and 2 illustrate the structures of a representative number of MFP compounds of the present invention. FIG. 1 also sets forth a reaction scheme that can be used to prepare the MFP compounds of the present invention. As illustrated in FIG. 1, starting from a symmetrical polyhydroxylated compound (1), the hydrophobic domain is added by, e.g., acylation of three of the four alcohols, resulting in a triester (2). The remaining alcohol is then converted to an acrylate (3) by adding acryloyl chloride. The polyamine moiety is then introduced by adding an excess of, e.g., N,N,N′-trimethylpropylenediamine to the acrylate (3), to yield the polyamine (4). The structure can be further elaborated by adding, e.g., methyl iodide, to produce the corresponding bis-quaternary ammonium salts (5).

It will be readily apparent to those of skill in the art that modifications can be made to the foregoing process to generate the other MFP compounds of the present invention. For instance, by adjusting the stoichiometry of a reagent in the synthetic steps or altering commercially available reagents one can produce MFP compounds having different chemical structures. Again, FIG. 2 illustrates the structures of a representative number of MFP compounds of the present invention that can be made using the reaction scheme set forth in FIG. 1.

C. Liposome Preparation and Composition

In an other aspect, the present invention relates to a transfection complex, i.e., a lipid-nucleic acid complex or lipoplex, comprising a nucleic acid and a MFP compound of Formula I. As indicated above, the methods of this invention involve complexing a nucleic acid with a compound of Formula I.

In certain aspects, the formulations further comprise a cationic lipid. The term “cationic lipid” refers to any of a number of lipid species which carry a net positive charge at physiological pH. Such lipids include, but are not limited to, DODAC, DOTMA, DDAB, DOTAP, DC-Chol and DMRIE. Additionally, a number of commercial preparations of cationic lipids are available which can be used in the present invention. These include, for example, LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic lipids comprising DOGS in ethanol from Promega Corp., Madison, Wis., USA).

The cationic lipid can be used alone, or in combination with a “helper” lipid. Preferred helper lipids are nonionic or uncharged at physiological pH. Particularly preferred nonionic lipids include, but are not limited to, DOPE, DOSPA, DMDHP, DOPC, cholesterol and mixtures thereof, with DOPE and cholesterol being most preferred. The molar ratio of cationic lipid to helper lipid can range from 2:1 to about 1:2, more preferably from about 1.5:1 to about 1:1.5 and most preferably is about 1:1.

In addition, the cationic lipids of this invention can be formulated into liposomes. Liposomes are constructed by well known techniques, such as described in Liposome Technology, Vols. 1-3 (G. Gregoriadis, Ed., CRC Press, 1993). Lipids are typically dissolved in chloroform and spread in a thin film over the surface of a tube or flask by rotary evaporation. If liposomes comprised of a mixture of lipids are desired, the individual components are mixed in the original chloroform solution. After the organic solvent has been eliminated, a phase consisting of water optionally containing buffer and/or electrolyte is added and the vessel agitated to suspend the lipid. Optionally, the suspension is then subjected to ultrasound, either in an ultrasonic bath or with a probe sonicator, until the particles are reduced in size and the suspension is of the desired clarity. For transfection, the aqueous phase is typically distilled water and the suspension is sonicated until nearly clear, which requires some minutes depending upon conditions, kind, and quality of the sonicator. Commonly, lipid concentrations are 1 mg/mL of aqueous phase, but could easily be higher or lower by a factor of ten.

The transfection complexes of the present invention comprise one or more of the MFP compounds, i.e., cationic lipids, of Formula I. In a preferred embodiment, the transfection complex is a lipoplex or a liposome. The transfection complexes of the present invention comprise at least one MFP compound of the present invention. In a preferred embodiment, the transfection complexes of the present invention comprise a single type of lipid of Formula I. In another preferred embodiment, the transfection complexes comprise mixtures of compounds of Formula I. In another preferred embodiment, the transfection complexes according to the invention optionally have one or more other lipids. As such, in a preferred embodiment, the liposomes of the present invention comprise one or more lipids of Formula I in a mixture with one or more natural or synthetic lipids, e.g., cholesterol or DOPE. The exact composition of the transfection complexes will depend on the particular circumstances for which they are to be used. Those of ordinary skill in the art will find it a routine matter to determine a suitable composition.

In a preferred embodiment, the transfection complexes of the present invention are liposomes. In a preferred embodiment, unilamellar liposomes are produced by the reverse phase evaporation method of Szoka & Papahadjopoulos, Proc. Natl. Acad. Sci. USA, 75: 4194-4198 (1978). Unilamellar vesicles are generally prepared by sonication or extrusion. Sonication is generally performed with a bath-type sonifier, such as a Branson tip sonifier at a controlled temperature as determined by the melting point of the lipid. Extrusion may be carried out by biomembrane extruders, such as the Lipex Biomembrane Extruder. Defined pore size in the extrusion filters may generate unilamellar liposomal vesicles of specific sizes. The liposomes may also be formed by extrusion through an asymmetric ceramic filter, such as a Ceraflow Microfilter, commercially available from the Norton Company, Worcester, Mass.

Following liposome preparation, the liposomes that have not been sized during formation may be sized by extrusion to achieve a desired size range and relatively narrow distribution of liposome sizes. A size range of about 0.2-0.4 microns allows the liposome suspension to be sterilized by filtration through a conventional filter, typically a 0.22 micron filter. The filter sterilization method can be carried out on a high throughput basis if the liposomes have been sized down to about 0.2-0.4 microns.

Several techniques are available for sizing liposomes to a desired size. One sizing method is described in U.S. Pat. Nos. 4,529,561 or 4,737,323, herein incorporated by reference. Sonicating a liposome suspension either by bath or probe sonication produces a progressive size reduction down to small unilamellar vesicles less than about 0.05 microns in size. Homogenization is another method which relies on shearing energy to fragment large liposomes into smaller ones. In a typical homogenization procedure, multilamellar vesicles are recirculated through a standard emulsion homogenizer until selected liposome sizes, typically between about 0.1 and 0.5 microns, are observed. The size of the liposomal vesicles may be determined by quasi-electric light scattering (QELS) as described in Bloomfield, Ann. Rev. Biophys. Bioeng., 10: 421-450 (1981). Average liposome diameter may be reduced by sonication of formed liposomes. Intermittent sonication cycles may be alternated with QELS assessment to guide efficient liposome synthesis.

Extrusion of liposomes through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes, to achieve a gradual reduction in liposome size. For use in the present invention, liposomes having a size of about 0.05 microns to about 0.5 microns. More preferred are liposomes having a size of about 0.05 to 0.2 microns.

D. Nucleic Acid

Nucleic acids of all types may be associated with the transfection complexes of the present invention and subsequently can be transfected. These include DNA, RNA, DNA/RNA hybrids (each of which may be single or double stranded), including oligonucleotides such as antisense oligonucleotides, chimeric DNA-RNA polymers, ribozymes, as well as modified versions of these nucleic acids, wherein the modification may be in the base, the sugar moiety, the phosphate linkage, or in any combination thereof.

Further, DNA may be in the form of antisense, plasmid DNA, parts of a plasmid DNA, product of a polymerase chain reaction (PCR), vectors (P1, PAC, BAC, YAC, artificial chromosomes), expression cassettes, chimeric sequences, chromosomal DNA, or derivatives of these groups. RNA may be in the form of oligonucleotide RNA, tRNA (transfer RNA), snRNA (small nuclear RNA), rRNA (ribosomal RNA), mRNA (messenger RNA), antisense RNA, siRNA (small interfering RNA), ribozymes, chimeric sequences, or derivatives of these groups.

From the foregoing it will be clear to those skilled in the art that the transfection complexes such as for example, lipoplexes, liposomes, and the like of the present invention are useful for both in vitro and in vivo application. The transfection complexes of the present invention will find use for nearly any in vitro application requiring transfection of nucleic acids into cells. For example, the process of recombinant production of a protein.

The nucleic acids may comprise an essential gene or fragment thereof, in which the target cell or cells is deficient in some manner. This can occur where the gene is lacking or where the gene is mutated resulting in under- or over-expression. The nucleic acids can also comprise antisense oligonucleotides. Such antisense oligonucleotides may be constructed to inhibit expression of a target gene. The foregoing are examples of nucleic acids that may be used with the present invention, and should not be construed to limit the invention in any way. Those skilled in the art will appreciate that other nucleic acids will be suitable for use in the present invention as well.

E. Method for Transfecting

In yet another aspect, this invention relates to a method for transfecting a nucleic acid into a cell. The method involves contacting a cell with a transfection complex comprising a nucleic acid and a compound of Formula I. Liposome-nucleic acid complex/aggregates may be prepared by adding an appropriate amount of nucleic acid to a liposome solution. For transfection, MFP head group to nucleic acid phosphates (N/P) is about 2.5:1 to about 10:1. The amount of DNA can vary considerably, but is normally a few to a few tens of micrograms per standard culture dish of cells. Conditions may vary widely, and it is a routine matter and standard practice to optimize conditions for each type of cell, as suppliers of commercial materials recommend. Optimization involves varying the lipid to DNA ratio as well as the total amount of aggregate.

There is currently some uncertainty regarding the precise way in which nucleic acids and cationic lipids interact. In addition, the structure formed both before and during the transfection process is not definitively known. The present invention, however, is not limited by the particular structural type of complex formed by the liposomes and lipid aggregates of the present invention and the nucleic acids to be transfected. The phrase “transfection complex” refers to any association of liposome, cationic lipid or lipid complex, and nucleic acid that is capable of lipofection.

In one embodiment, the transfection complex is added to the cells, in culture medium, or administered to cells in vivo. When added to cells in vitro, the transfection complex is added to cells and left for some tens of minutes to several hours to perhaps overnight. Usually serum is omitted from the culture medium during this phase of transfection. Subsequently, the medium is replaced with normal, serum-containing medium and the cells are incubated for hours to days or possibly cultured indefinitely.

F. Specific Target Tissues

In certain embodiments, it may be desirable to use specific targeting moieties with the transfection complexes of the present inveniton to target specific cells or tissues. In one embodiment, the targeting moiety, such as an antibody or antibody fragment, is attached to a hydrophilic polymer and is combined with the transfection complex, i.e., the lipid:nucleic acid complex, after complex formation. Thus, the use of a targeting moiety in combination with a generic effector lipid:nucleic acid complex provides the ability to conveniently customize the complex for delivery to specific cells and tissues.

Examples of effectors in lipid:nucleic acid complexes include nucleic acids encoding cytotoxins (e.g., diphtheria toxin (DT), Pseudomonas exotoxin A (PE), pertussis toxin (PT), and the pertussis adenylate cyclase (CYA)), antisense nucleic acid, ribozymes, labeled nucleic acids, and nucleic acids encoding tumor suppressor genes such as p53, p110Rb, and p72. These effectors can be specifically targeted to cells such as cancer cells, immune cells (e.g., B and T cells), and other desired cellular targets with a targeting moiety. For example, as described above, many cancers are characterized by overexpression of cell surface markers such as HER2, which is expressed in breast cancer cells, or IL17R, which is expressed in gliomas. Targeting moieties such as anti-HER2 and anti-IL17R antibodies or antibody fragments are used to deliver the lipid:nucleic acid complex to the cell of choice. The effector molecule is thus delivered to the specific cell type, providing a useful and specific therapeutic treatment.

G. Drug Delivery

In still yet another aspect, this invention relates to a pharmaceutical composition or other drug delivery composition for administering a transfection complex to a cell. This composition includes a transfection complex comprising a nucleic acid and MFP compound of Formula I, and a pharmaceutically acceptable carrier therefor. As used herein, the term “pharmaceutical composition” means any association of a MFP compound of Formula I and a nucleic acid and or a mixture of a conventional drug capable of being delivered into cells.

Cationic lipid-assisted drug delivery may be accomplished in the following manner. For drugs that are soluble in organic solvents, such as chloroform, the drug and cationic lipid are mixed in solvents in which both are soluble, and the solvent is then removed under vacuum. The lipid-drug residue is then dispersed in an appropriate aqueous solvent, which, in a preferred embodiment, is sterile physiological saline. The suspension then may optionally be subjected to up to several freeze/thaw cycles. It is then sonicated, either merely to reduce the coarseness of the dispersion or to reduce the particle size to 20-30 nm diameter, depending upon whether large or small particle size is most efficacious in the desired application. For some applications, it may be most effective to generate extruded liposomes by forming the suspension through a filter with pores of 100 nm diameter or smaller. For some applications, inclusion of cholesterol or natural phospholipids in the mixture used to generate the lipid-drug complex can be appropriate.

The liposome-drug complex may then be delivered in any suitable manner. For drugs that are soluble in aqueous solution and insoluble in organic solvents, the lipid mixture to be used for the lipid dispersion or liposomes is coated on the inside surface of a flask or tube by evaporating the solvent from a solution of the mixture. In general, for this method to be successful, the lipid mixture must be capable of forming vesicles having single or multiple lipid bilayer walls and encapsulating an aqueous core. The aqueous phase containing the dissolved drug, preferably a physiological saline solution, is added to the lipid, agitated to generate a suspension, and then optionally frozen and thawed up to several times.

To generate small liposomes the suspension is subjected, for example, to ultrasonic waves for a time necessary to reduce the liposomes to the desired average size. If large liposomes are desired, the suspension is merely agitated by hand or on a vortex mixer until a uniform dispersion is obtained, i.e., until visually observable large particles are absent. If the preparation is to have the drug contained only within the liposomes, then the drug in the aqueous phase is eliminated by dialysis or by passage through a gel-filtration chromatographic column (e.g., agarose) equilibrated with the aqueous phase containing all normal components except the drug. The lipid mixture used can contain cholesterol or natural lipids in addition to the cationic compounds of the present invention. The liposome-drug aggregate may then be delivered in any suitable manner.

H. Disease Treatment

In yet another aspect of the invention comprises novel methods of treating diseases arising from infection by a pathogen or from an endogenous DNA deficiency. These methods comprise administering a transfection complex and/or liposome-drug aggregate solution to a mammal suffering from a pathogenic infection or DNA deficiency. If the disease is the result of infection by a pathogen, the nucleic acid can be an antisense oligonucleotide targeted against a DNA sequence in the pathogen that is essential for development, metabolism, or reproduction of the pathogen. If the disease is a DNA deficiency (i.e., wherein certain endogenous DNA is missing or has been mutated), resulting in under- or over-expression, the nucleic acid maybe the normal DNA sequence.

Several methods of in vivo lipofection have been reported. In the case of whole animals, the transfection complex may be injected into the blood stream, directly into a tissue, into the peritoneum, instilled into the trachea, or converted to an aerosol, which the animal breathes. Zhu et al., Science, 261:209-211 (1993), describe a single intravenous injection of 100 micrograms of a mixture of DNA and DOTMA:dioleoylphosphatidylethanaolamine that efficiently transfected virtually all tissues. It is also possible to use a catheter to implant liposome-DNA aggregates in a blood vessel wall, which can result in successful transformation of several cell types, including endothelial and vascular smooth muscle cells. Stribling et al., Proc. Natl. Acad. Sci. USA, 89:11277-11281 (1992), demonstrated that aerosol delivery of a chloramphenicol acetyltransferase (CAT) expression plasmid complexed to cationic liposomes produced high-level, lung-specific CAT gene expression in mice in vivo for at least 21 days. They described the following procedure: Six milligrams of plasmid DNA and 12 μmol of DOTMA/DOPE liposomes were each diluted to 8 mL with water and mixed; equal volumes were then placed into two Acorn I nebulizers (Marquest, Englewood, Colo.); animals were loaded into an Intox small-animal exposure chamber (Albuquerque) and an air flow rate of 4L/min was used to generate the aerosol (about 90 min were required to aerosolize this volume) the animals were removed from the chamber for 1-2 hours and the procedure was repeated. This protocol is representative of the aerosol delivery method.

### EXAMPLES

### Example I

### Preparation of Transfection Complexes

Compounds 4 and 5 of FIG. 1 were blended with DOPE or cholesterol and then hydrated to form aqueous suspensions. These aqueous suspensions were mixed with plasmid DNA to form an active complex that can be administered in vitro or in vivo, thereby resulting in increased levels of transgene expression relative to administration of “free” plasmid DNA.

### Example II

### Transfection of NIH-3T3 Cells with Compounds 1 and 2

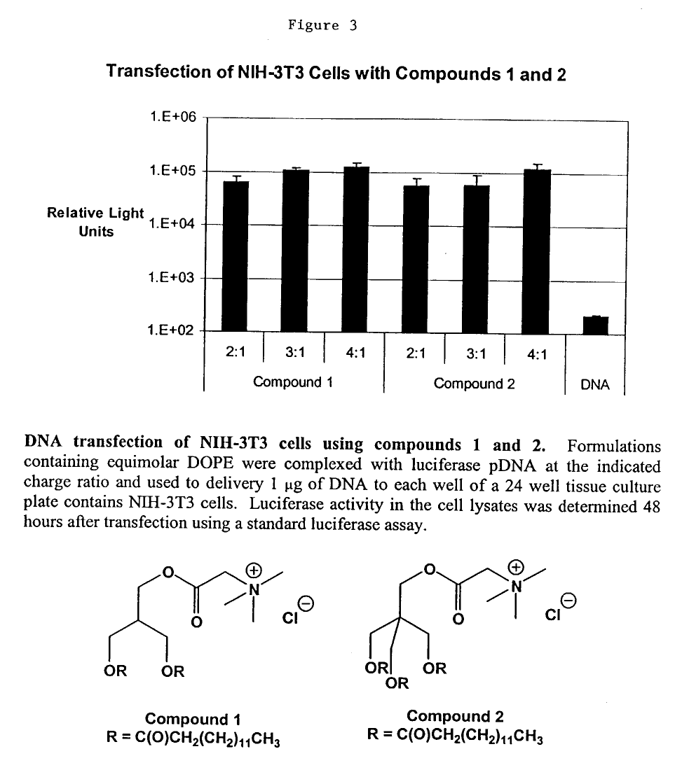

Formulations containing equimolar amounts of DOPE and compounds 1 and 2, respectively, which are illustrated in FIG. 3, were complexed with luciferase pDNA at the indicated charge ratio and used to deliver 1 μg of DNA to each well of a 24-well tissue culture plate containing NIH-3T3 cells. Luciferase activity in the cell lysates was determined 48 hours after transfection using a standard luciferase assay. Results obtained using Compounds 1 and 2 of the present invention and “free” DNA are shown in FIG. 3.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated herein by reference for all purpose.

What is claimed is:

1. A compound having the formula 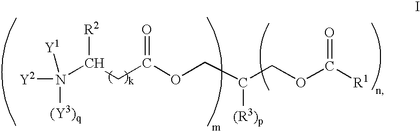
or a pharmaceutically acceptable salt thereof, wherein:
each R¹ is independently selected from the group consisting of optionally substituted C₈-C₂₄ alkyl and optionally substituted C₈-C₂₄ alkenyl;
each R² and R³ is independently selected from the group consisting of hydrogen, optionally substituted C₁-C₈ alkyl, optionally substituted arylalkyl, optionally substituted C₁-C₈ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl;
each Y¹ and Y² is independently selected from the group consisting of hydrogen, optionally substituted C₁-C₆ alkyl, optionally substituted arylalkyl, optionally substituted C₁-C₆ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl;
each Y³, if present, is independently selected from the group consisting of hydrogen, optionally substituted C₁-C₈ alkyl, optionally substituted arylalkyl, optionally substituted C₁-C₈ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl;
m is an integer selected from the group consisting of 1, 2, 3 and 4, n is an integer selected from the group consisting of 0, 1, 2 and 3, and p is an integer selected from the group consisting of 0 and 1, wherein the sum of m, n and p is 4;
each k is an integer independently selected from the group consisting of 1, 2, 3, 4 and 5; and
each q is an integer independently selected from the group consisting of 0 and 1;
or, each R², Y¹ and the atoms to which they are bound, join to form an optionally substituted 5- or 6-membered heterocyclic ring;
with the proviso that if m is 2, n is 2, q is 0 and Y¹ is hydrogen, then Y² is not an optionally substituted aminoalkyl.

2. A compound in accordance with claim 2, wherein each q is 1 and said compound is a pharmaceutically acceptable salt.

3. A compound in accordance with claim 2, wherein said pharmaceutically acceptable salt is at least one quaternary nitrogen salt selected from the group consisting of a quaternary ammonium chloride, a quaternary ammonium iodide, a quaternary ammonium fluoride, a quaternary ammonium bromide, a quaternary ammonium oxyanion and a combination thereof.

4. A compound in accordance with claim 2, wherein said pharmaceutically acceptable salt is a quaternary ammonium iodide.

5. A compound in accordance with claim 1, wherein:
each R¹ is a member selected from the group consisting of myristyl, oleyl, lauryl and palmityl.

6. A compound in accordance with claim 1 wherein:
each R² and R³ is a member independently selected from the group consisting of hydrogen, methyl, ethyl, isopropyl, isobutyl, secbutyl, hydroxymethyl, thiomethyl, carboxymethyl, guanidinopropyl, carbamoylmethyl, carbamoylethyl, benzyl, p-hydroxyphenylmethyl, 1-hydroxyethyl, 2-(methylthio)ethyl, 3-indolemethyl, carboxyethyl and aminobutyl.

7. A compound in accordance with claim 1, wherein:
each R² and R³ is a member independently selected from the group consisting of hydrogen, methyl and isopropyl.

8. A compound in accordance with claim 1, wherein:
each Y¹ and Y³ is a member independently selected from the group consisting of methyl, ethyl, propyl and butyl; and
each Y² is a member independently selected from the group consisting of N,N-dimethylethylamine, N,N-dimethylpropylamine and N,N-dimethylbutylamine.

9. A compound in accordance with claim 1, wherein at least one R², Y¹ and the atoms to which they are bound, join to form a member selected from the group consisting of a pyrrolidine ring, a 4-hydroxypyrrolidine ring and an imidazolylmethyl ring.

10. A compound in accordance with claim 1, wherein:
each R¹ is a member independently selected from the group consisting of myristyl and oleyl;
each R² and R³ is hydrogen;
each Y¹ and Y³ is methyl; and
each Y² is a member independently selected from the group consisting of N,N-dimethylethylamine, N,N-dimethylpropylamine and N,N-dimethylbutylamine.

11. A compound in accordance with claim 10, wherein m is 1, n is 3, p is 0 and q is 0 or 1.

12. A compound in accordance with claim 10, wherein m is 3, n is 1, p is 0 and q is 0 or 1.

13. A compound in accordance with claim 10, wherein m is 2, n is 2, p is 0 and q is 0 or 1.

14. A compound in accordance with claim 10, wherein m is 1, n is 2, p is 1 and q is 0 or 1.

15. A transfection complex, said transfection complex comprising a nucleic acid and a compound of Formula I: 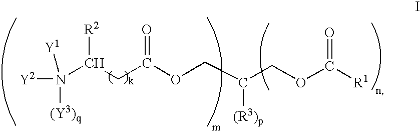
or a pharmaceutically acceptable salt thereof, wherein:
each R¹ is independently selected from the group consisting of optionally substituted C₈-C₂₄ alkyl and optionally substituted C₈-C₂₄ alkenyl;
each R² and R³ is independently selected from the group consisting of hydrogen, optionally substituted C₁-C₈ alkyl, optionally substituted arylalkyl, optionally substituted C₁-C₈ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl;
each Y¹ and Y² is independently selected from the group consisting of hydrogen, optionally substituted C₁-C₆ alkyl, optionally substituted arylalkyl, optionally substituted C₁-C₆ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl;
each Y³, if present, is independently selected from the group consisting of hydrogen, optionally substituted C₁-C₈ alkyl, optionally substituted arylalkyl, optionally substituted C₁-C₈ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl;
m is an integer selected from the group consisting of 1, 2, 3 and 4, n is an integer selected from the group consisting of 0, 1, 2 and 3, and p is an integer selected from the group consisting of 0 and 1, wherein the sum of m, n and p is 4;
each k is an integer independently selected from the group consisting of 1, 2, 3, 4 and 5; and each q is an integer independently selected from the group consisting of 0 and 1;
or, each R², Y¹ and the atoms to which they are bound, join to form an optionally substituted 5- or 6-membered heterocyclic ring,
with the proviso that if m is 2, n is 2, q is 0 and Y¹ is hydrogen, then Y² is not an optionally substituted aminoalkyl.

16. A transfection complex in accordance with claim 15, wherein each q is 1 and said compound is a pharmaceutically acceptable salt.

17. A transfection complex in accordance with claim 16, wherein said pharmaceutically acceptable salt is at least one quaternary nitrogen salt selected from the group consisting of a quaternary ammonium chloride, a quaternary ammonium iodide, a quaternary ammonium fluoride, a quaternary ammonium bromide, a quaternary ammonium oxyanion and a combination thereof.

18. A transfection complex in accordance with claim 15, wherein:
each R¹ is a member selected from the group consisting of myristyl, oleyl, lauryl and palmityl.

19. A transfection complex in accordance with claim 15, wherein:
each R² and R³ is a member independently selected from the group consisting of hydrogen, methyl, ethyl, isopropyl, isobutyl, secbutyl, hydroxymethyl, thiomethyl, carboxymethyl, guanidinopropyl, carbamoylmethyl, carbamoylethyl, benzyl, p-hydroxyphenylmethyl, 1-hydroxyethyl, 2-(methylthio)ethyl, 3-indolemethyl, carboxyethyl and aminobutyl.

20. A transfection complex in accordance with claim 15, wherein:
each R² and R³ is a member independently selected from the group consisting of hydrogen, methyl and isopropyl.

21. A transfection complex in accordance with claim 15, wherein:
each Y¹ and Y³ is a member independently selected from the group consisting of methyl, ethyl, propyl and butyl; and
each Y² is a member independently selected from the group consisting of N,N-dimethylethylamine, N,N-dimethylpropylamine and N,N-dimethylbutylamine.

22. A transfection complex in accordance with claim 15, wherein each R², Y¹ and the atoms to which they are bound, join to form a member independently selected from the group consisting of a pyrrolidine ring, a 4-hydroxypyrrolidine ring and an imidazolylmethyl ring.

23. A transfection complex in accordance with claim 15, wherein:
each R¹ is a member selected from the group consisting of myristyl and oleyl;
each R² and R³ is hydrogen;
each Y¹ and Y³ is methyl; and
each Y² is a member independently selected from the group consisting of N,N-dimethylethylamine, N,N-dimethylpropylamine and N,N-dimethylbutylamine.

24. A transfection complex in accordance with claim 23, wherein m is 1, n is 3, p is 0 and q is 0 or 1.

25. A transfection complex in accordance with claim 23, wherein m is 3, n is 1, p is 0 and q is 0 or 1.

26. A transfection complex in accordance with claim 23, wherein m is 2, n is 2, p is 0 and q is 0 or 1.

27. A transfection complex in accordance with claim 23, wherein m is 1, n is 2, p is 1 and q is 0 or 1.

28. A transfection complex in accordance with claim 15, wherein said nucleic acid is RNA or DNA.

29. A transfection complex in accordance with claim 15, wherein said nucleic acid is plasmid DNA.

30. A transfection complex in accordance with claim 15, wherein said nucleic acid is antisense RNA or DNA.

31. A transfection complex in accordance with claim 23, wherein said transfection complex further comprises a second lipid.

32. A transfection complex in accordance with claim 23, wherein said second lipid is a member selected from the group consisting of DOSPA, DOPE, DMDHP, cholesterol and combinations thereof.

33. A method for transfecting a cell with a nucleic acid, said method comprising contacting said cell with a transfection complex comprising said nucleic acid and a compound of Formula I: 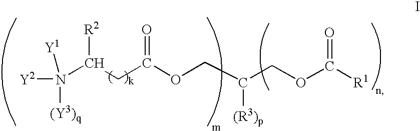
or a pharmaceutically acceptable salt thereof, wherein:
each R¹ is independently selected from the group consisting of optionally substituted C₈-C₂₄ alkyl and optionally substituted C₈-C₂₄ alkenyl;
each R² and R³ is independently selected from the group consisting of hydrogen, optionally substituted C₁-C₈ alkyl, optionally substituted arylalkyl, optionally substituted C₁-C₈ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl;
each Y¹ and Y² is independently selected from the group consisting of hydrogen, optionally substituted C₁-C₆ alkyl, optionally substituted arylalkyl, optionally substituted C₁-C₆ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl;
each Y³, if present, is independently selected from the group consisting of hydrogen, optionally substituted C₁-C₈ alkyl, optionally substituted arylalkyl, optionally substituted C₁-C₈ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl;
m is an integer selected from the group consisting of 1, 2, 3 and 4, n is an integer selected from the group consisting of 0, 1, 2 and 3, and p is an integer selected from the group consisting of 0 and 1, wherein the sum of m, n and p is 4;
each k is an integer independently selected from the group consisting of 1, 2, 3, 4 and 5; and
each q is an integer independently selected from the group consisting of 0 and 1;
or, each R², Y¹ and the atoms to which they are bound, join to form an optionally substituted 5- or 6-membered heterocyclic ring, with the proviso that if m is 2, n is 2, q is 0 and Y¹ is hydrogen, then Y² is not an optionally substituted aminoalkyl.

34. A method for transfecting a cell with a nucleic acid in accordance with claim 33, wherein each q is 1 and said compound is a pharmaceutically acceptable salt.

35. A method for transfecting a cell with a nucleic acid in accordance with claim 34, wherein said pharmaceutically acceptable salt is at least one quaternary nitrogen salt selected from the group consisting of a quaternary ammonium chloride, a quaternary ammonium iodide, a quaternary ammonium fluoride, a quaternary ammonium bromide, a quaternary ammonium oxyanion and a combination thereof.

36. A method for transfecting a cell with a nucleic acid in accordance with claim 33, wherein:
each R¹ is a member independently selected from the group consisting of myristyl, oleyl, lauryl and palmityl.

37. A method for transfecting a cell with a nucleic acid in accordance with claim 33, wherein:
each R² and R³ is a member independently selected from the group consisting of hydrogen, methyl, ethyl, isopropyl, isobutyl, secbutyl, hydroxymethyl, thiomethyl, carboxymethyl, guanidinopropyl, carbamoylmethyl, carbamoylethyl, benzyl, p-hydroxyphenylmethyl, 1-hydroxyethyl, 2-(methylthio)ethyl, 3-indolemethyl, carboxyethyl and aminobutyl.

38. A method for transfecting a cell with a nucleic acid in accordance with claim 33, wherein:
each R² and R³ is a member independently selected from the group consisting of hydrogen, methyl and isopropyl.

39. A method for transfecting a cell with a nucleic acid in accordance with claim 33, wherein:
each Y¹ and Y³ is a members independently selected from the group consisting of methyl, ethyl, propyl and butyl; and
each Y² is a member independently selected from the group consisting of N,N-dimethylethylamine, N,N-dimethylpropylamine and N,N-dimethylbutylamine.

40. A method for transfecting a cell with a nucleic acid in accordance with claim 33, wherein at least one R², Y¹ and the atoms to which they are bound, join to form a member selected from the group consisting of a pyrrolidine ring, a 4-hydroxypyrrolidine ring and an imidazolylmethyl ring.

41. A method for transfecting a cell with a nucleic acid in accordance with claim 33, wherein:
each R¹ is a member selected from the group consisting of myristyl and oleyl;
each R² and R³ is hydrogen;
each Y¹ and Y³ is methyl; and
each Y² is a member independently selected from the group consisting of N,N-dimethylethylamine, N,N-dimethylpropylamine and N,N-dimethylbutylamine.

42. A method for transfecting a cell with a nucleic acid in accordance with claim 41, wherein m is 1, n is 3, p is 0 and q is 0 or 1.

43. A method for transfecting a cell with a nucleic acid in accordance with claim 41, wherein m is 3, n is 1, p is 0 and q is 0 or 1.

44. A method for transfecting a cell with a nucleic acid in accordance with claim 41, wherein m is 2, n is 2, p is 0 and q is 0 or 1.

45. A method for transfecting a cell with a nucleic acid in accordance with claim 41, wherein m is 1, n is 2, p is 1 and q is 0 or 1.

46. A method for transfecting a cell with a nucleic acid in accordance with claim 33, wherein said nucleic acid is RNA or DNA.

47. A method for transfecting a cell with a nucleic acid in accordance with claim 33, wherein said transfection complex further comprises a second lipid.

48. A pharmaceutical composition comprising:
(a) a transfection complex comprising a nucleic acid and a compound of Formula I: 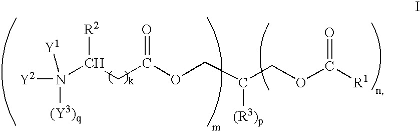
or a pharmaceutically acceptable salt thereof, wherein:
each R¹ is independently selected from the group consisting of optionally substituted C₈-C₂₄ alkyl and optionally substituted C₈-C₂₄ alkenyl;
each R² and R³ is independently selected from the group consisting of hydrogen, optionally substituted C₁-C₈ alkyl, optionally substituted arylalkyl, optionally substituted C₁-C₈ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl;
each Y¹ and Y² is independently selected from the group consisting of hydrogen, optionally substituted C₁-C₆ alkyl, optionally substituted arylalkyl, optionally substituted C₁-C₆ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl;
each Y³, if present, is independently selected from the group consisting of hydrogen, optionally substituted C₁-C₈ alkyl, optionally substituted arylalkyl, optionally substituted C₁-C₈ alkylthioalkyl, optionally substituted guanidinoalkyl, optionally substituted carboxyalkyl, optionally substituted aminoalkyl, optionally substituted carbamoyl C₁-C₈ alkyl and optionally substituted heteroarylalkyl;
m is an integer selected from the group consisting of 1, 2, 3 and 4, n is an integer selected from the group consisting of 0, 1, 2 and 3, and p is an integer selected from the group consisting of 0 and 1, wherein the sum of m, n and p is 4;
each k is an integer independently selected from the group consisting of 1, 2, 3, 4 and 5; and
each q is an integer independently selected from the group consisting of 0 and 1;
or, each R², Y¹ and the atoms to which they are bound, join to form an optionally substituted 5- or 6-membered heterocyclic ring,
with the proviso that if m is 2, n is 2, q is 0 and Y¹ is hydrogen, then Y² is not an optionally substituted aminoalkyl; and
(b) a pharmaceutically acceptable carrier.

49. A pharmaceutical composition in accordance with claim 49, wherein each q is 1 and said compound is a pharmaceutically acceptable salt.

50. A pharmaceutical composition in accordance with claim 49, wherein said pharmaceutically acceptable salt is at least one quaternary nitrogen salt selected from the group consisting of a quaternary ammonium chloride, a quaternary ammonium iodide, a quaternary ammonium fluoride, a quaternary ammonium bromide, a quaternary ammonium oxyanion and a combination thereof.

51. A pharmaceutical composition in accordance with claim 49, wherein:
each R¹ is a member independently selected from the group consisting of myristyl, oleyl, lauryl and palmityl.

52. A pharmaceutical composition in accordance with claim 49, wherein:
each R² and R³ is a member independently selected from the group consisting of hydrogen, methyl, ethyl, isopropyl, isobutyl, secbutyl, hydroxymethyl, thiomethyl, carboxymethyl, guanidinopropyl, carbamoylmethyl, carbamoylethyl, benzyl, p-hydroxyphenylmethyl, 1-hydroxyethyl, 2-(methylthio)ethyl, 3-indolemethyl, carboxyethyl and aminobutyl.

53. A pharmaceutical composition in accordance with claim 49, wherein:
each R² and R³ is a member independently selected from the group consisting of hydrogen, methyl and isopropyl.

54. A pharmaceutical composition in accordance with claim 49, wherein:
each Y¹ and Y³ is a member independently selected from the group consisting of methyl, ethyl, propyl and butyl; and
each Y² is a member independently selected from the group consisting of N,N-dimethylethylamine, N,N-dimethylpropylamine and N,N-dimethylbutylamine.

55. A pharmaceutical composition in accordance with claim 49, wherein at least one R², Y¹ and the atoms to which they are bound, join to form a member selected from the group consisting of a pyrrolidine ring, a 4-hydroxypyrrolidine ring and an imidazolylmethyl ring.

56. A pharmaceutical composition in accordance with claim 49, wherein:
each R¹ is a member independently selected from the group consisting of myristyl and oleyl;
each R² and R³ is hydrogen;
each Y¹ and Y³ is methyl; and
each Y² is a member independently selected from the group consisting of N,N-dimethylethylamine, N,N-dimethylpropylamine and N,N-dimethylbutylamine.

57. A pharmaceutical composition in accordance with claim 56, wherein m is 1, n is 3, p is 0 and q is 0 or 1.

58. A pharmaceutical composition in accordance with claim 56, wherein m is 3, n is 1, p is 0 and q is 0 or 1.

59. A pharmaceutical composition in accordance with claim 56, wherein m is 2, n is 2, p is 0 and q is 0 or 1.

60. A pharmaceutical composition in accordance with claim 56, wherein m is 1, n is 2, p is 1 and q is 0 or 1.

61. A pharmaceutical composition in accordance with claim 49, wherein said nucleic acid is DNA or RNA.

62. A pharmaceutical composition in accordance with claim 49, wherein said transfection complex further comprises a second lipid.
